(19) United States
(12) Patent Application Publication
(10) Pub. No.: US 2004/0203006 A1
(43) Pub. Date: Oct. 14, 2004

(54) Novel beta-tubulin protein of Candida glabrata and methods for its use

(75) Inventors: Mary Maxon, San Francisco, CA (US); David Clarke, San Francisco, CA (US); Christophe Beraud, Palm Springs, CA (US)

Correspondence Address: FINNEGAN, HENDERSON, FARABOW, GARRETT & DUNNER, LLP, 1300 I STREET, NW, WASHINGTON, DC, 20005, US

(73) Assignee: Cytokinetics, Inc.

(21) Appl. No.: 10/412,439

(22) Filed: Apr. 11, 2003

(51) Int. Cl. C12Q 1/68; C07H 21/04; C07K 14/47

(52) U.S. Cl. 435/6; 435/69.1; 435/320.1; 435/325; 530/358; 536/23.5

(57) ABSTRACT

The present invention provides isolated nucleic acid and amino acid sequences of Candida glabrata beta-tubulin, antibodies to C. glabrata beta-tubulin, and methods of screening to identify antifungals using biologically active C. glabrata beta-tubulin.

### FIELD OF THE INVENTION

The present invention provides isolated nucleic acid and amino acid sequences of Candida glabrata beta-tubulin, antibodies to C. glabrata beta-tubulin, and methods of screening to identify antifungals using biologically active C. glabrata beta-tubulin.

### BACKGROUND OF THE INVENTION

Microtubules are subcellular polymers found in most eukaryotic cells, and are a major component of the cytoskeleton. Microtubules play critical roles in numerous aspects of cell physiology, including mitosis, intracellular movement, cell movement, and maintenance of cell shape. Microtubule assembly involves polymerization of tubulin subunits and additional construction with microtubule-associated proteins. In cell division, microtubules are organized into a specialized essential structure called the mitotic spindle.

Tubulin protein is a key component of microtubules. Tubulin consists of two 50 kDa subunits, designated as alpha.- and beta.-tubulin, which combine to form a heterodimer. The heterodimer binds two molecules of GTP. One GTP molecule is tightly bound, and can only be removed by denaturing the heterodimer. The other GTP molecule is freely exchangeable with other GTP molecules. The exchangeable GTP is believed to be involved in tubulin function. The tubulin heterodimer can combine in the presence of GTP to form a protofilament. These protofilaments can then group together to form a microtubule. Any interference with the construction of microtubules would be expected to be detrimental to the mitotic process.

Candida is a yeast, and the most common cause of opportunistic mycoses world-wide. Candida species are the most important fungal pathogens of humans, causing primarily mucosal infections, which in immunocompromised patients can breach the mucosal barrier and cause life-threatening systemic infections. The genus Candida includes around 154 species, 6 of which are most frequently isolated in human infections. While Candida albicans is the most abundant and significant species, Candida glabrata has frequently been isolated as a causative agent of Candida infections.

Recent reports have shown that there has been a recent increase in infections due to non-albicans Candida species such as Candida glabrata. See Pfaller, M. A., Epidemiology of Candidiasis. J. Hosp. Infeci., 1995, 30: 329-338. C. glabrata is currently the most frequently isolated fungal species in hospital intensive care units across the United States. Current estimates are that of species commonly causing invasive candidiasis, C. glabrata is responsible for a frequency approaching 30%. Immunocompromised patients receiving fluconazole prophylaxis are particularly at risk of developing infections due to fluconazole-resistant C. glabrata. Those at particular risk for such opportunistic infections are individuals with AIDS; those having undergone bone marrow or organ transplants; those receiving chemotherapy; and others who have had debilitating illness, severe injury, prolonged hospitalization, or long-term treatment with antibacterial drugs. Individuals with endocrinological conditions such as diabetes mellitus, hypoparathyroidism, or Addison's disease are also at high risk for C. glabrata infection.

The discovery of the Candida glabrata beta-tubulin protein, and the polynucleotides encoding it, satisfies a need in the art by providing new compositions which are useful in the development of treatments of immunocompromised patients afflicted with opportunistic C. glabrata-related fungal infections.

### SUMMARY OF THE INVENTION

The present invention is based on the discovery of a new beta-tubulin protein in C. glabrata, the polynucleotide encoding the beta-tubulin, and the use of these compositions for the diagnosis, treatment, or prevention of opportunistic fungal infections.

In one aspect, the invention provides an isolated nucleic acid sequence encoding a beta-tubulin protein, wherein the protein comprises an amino acid sequence that has greater than 70% amino acid sequence identity to SEQ ID NO: 2 as measured using a sequence comparison algorithm. In one embodiment, the protein further specifically binds to polyclonal antibodies raised against SEQ ID NO: 2.

In one embodiment, the nucleic acid encodes the C. glabrata beta-tubulin protein or a fragment thereof. In another embodiment, the nucleic acid encodes SEQ ID NO: 2. In another embodiment, the nucleic acid has a nucleotide sequence of SEQ ID NO: 1 or SEQ ID NO:3.

In one aspect, the nucleic acid comprises a sequence which encodes an amino sequence which has greater than 70% sequence identity with SEQ ID NO: 2, preferably greater than 80%, more preferably greater than 90%, more preferably greater than 95%, or, in another embodiment, has 98 to 100% sequence identity with SEQ ID NO: 2.

In one embodiment, the nucleic acid comprises a sequence which has greater than 55 or 60% sequence identity with SEQ ID NO: 1 or SEQ ID NO:3; preferably greater than 70%, more preferably greater than 80%, more preferably greater than 90 or 95%, or, in another embodiment, has 98 to 100% sequence identity with SEQ ID NO: 1 or SEQ ID NO:3. In another embodiment provided herein, the nucleic acid hybridizes under stringent conditions to a nucleic acid having a sequence of SEQ ID NO: 1 or SEQ ID NO:3.

In another aspect, the invention provides an expression vector, wherein the vector has one or more of the properties described above.

In a further aspect, the present invention provides a host cell transformed with the expression vector aforementioned.

In another embodiment, the present invention provides a method of identifying a compound as a modulator of an activity of the protein. The method comprises contacting the protein or a host cell containing the protein with a compound at a first concentration and determining a level of activity of the target protein. The method further comprises contacting the protein with a compound at a second concentration, and then determining a level of activity of the protein. A change in the level of activity between the protein contacted with the first concentration and the second concentration indicates that the compound modulates an activity of the protein.

Also provided are modulators of the protein including agents for the treatment of fungal infection as common in conditions of immunocompromise or impaired immunity, including AIDS, bone marrow or organ transplants, chemotherapy, or prolonged antibacterial therapy. The agents and compositions provided herein can be used in a variety of applications and formulations.

### BRIEF DESCRIPTION OF THE SEQUENCE LISTING

The Sequence Listing, which is incorporated herein by reference in its entirety, provides exemplary sequences including polynucleotide sequence SEQ ID NO: 1 or SEQ ID NO:3 (wherein SEQ ID NO:3 is the opening reading frame), and polypeptide sequence SEQ ID NO: 2.

### DETAILED DESCRIPTION OF THE INVENTION

Definitions

“Allele” refers to any of two or more alternative forms of a gene occupying the same chromosomal locus.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term antibody also includes antibody fragments either produced by the modification of whole antibodies or those synthesized de novo using recombinant DNA methodologies.

“Variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCT all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each degenerate codon in a nucleic acid can be modified to yield a functionally identical molecule. Accordingly, each silent variation of a nucleic acid which encodes a polypeptide is implicit in each described sequence.

Amino acid substitutions are typically of single residues; insertions usually will be on the order of from about 1 to about 20 amino acids, although considerably longer insertions may be tolerated. Deletions range from about 1 to about 20 residues, although in some cases, deletions may be much longer.

Substitutions, deletions, and insertions or any combinations thereof may be used to arrive at a final derivative. Generally, these changes are done on a few amino acids to minimize the alteration of the molecule. However, larger characteristics may be tolerated in certain circumstances.

The following six groups each contain amino acids that are conservative substitutions for one another:

Alanine (A), Serine (S), Threonine (T);

Aspartic acid (D), Glutamic acid (E);

Asparagine (N), Glutamine (Q);

Arginine (R), Lysine (K);

Isoleucine (I), Leucine (L), Methionine (M), Valine (V); and

Phenylalanine (F), Tyrosine (Y), Tryptophan (W).

(see, e.g., Creighton, Proteins (1984)).

“Cytoskeletal component” denotes any molecule that is found in association with the cellular cytoskeleton, that plays a role in maintaining or regulating the structural integrity of the cytoskeleton, or that mediates or regulates motile events mediated by the cytoskeleton. Includes cytoskeletal polymers (e.g., actin filaments, microtubules, intermediate filaments), molecular motors (e.g., kinesins, myosins, dyneins), cytoskeleton associated regulatory proteins (e.g., tropomysin, alpha-actinin) and cytoskeletal associated binding proteins (e.g., microtubules associated proteins, actin binding proteins).

“Cytoskeletal function” refers to biological roles of the cytoskeleton, including but not limited to the providing of structural organization (e.g., microvilli, mitotic spindle) and the mediation of motile events within the cell (e.g., muscle contraction, mitotic chromosome movements, contractile ring formation and function, pseudopodal movement, active cell surface deformations, vesicle formation and translocation.)

A “diagnostic” as used herein is a compound, method, system, or device that assists in the identification and characterization of a health or disease state. The diagnostic can be used in standard assays as is known in the art.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

“High stringency conditions” may be identified by those that: (1) employ low ionic strength and high temperature for washing, for example 0.2 times SSC and 0.1% Sodium dodecyl sulfate at 68. degree C.; (2) employ during hybridization a denaturing agent such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42.degree. C.; or (3) employ 50% formamide, 5.times.SSC (0.75 M NaCl, 0.075 M sodium citrate), 50 mM sodium phosphate (pH 6.8), 0.1% sodium pyrophosphate, 5.times.Denhardt's solution, sonicated salmon sperm DNA (50 .mu.g/ml), 0.1% SDS, and 10% dextran sulfate at 42.degree. C., with washes in 0.2.times.SSC (sodium chloride/sodium citrate) and 50% formamide at 55.degree. C., followed by a high-stringency wash consisting of 0.1.times.SSC containing EDTA at 55.degree. C.

A “host cell” is a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, or plant cells. Both primary cells and cultured cell lines are included in this definition.

The terms “isolated”, “purified”, or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In an isolated gene, the nucleic acid of interest is separated from open reading frames which flank the gene of interest and encode proteins other than the protein of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Modulators,” “inhibitors,” and “activators of a target protein” refer to modulatory molecules identified using in vitro and in vivo assays for target protein activity. Such assays include microtubule depolymerizing activity and binding activity such as microtubule binding activity or binding of nucleotide analogs. Samples or assays that are treated with a candidate agent at a test and control concentration. The control concentration can be zero. If there is a change in target protein activity between the two concentrations, this change indicates the identification of a modulator. A change in activity, which can be an increase or decrease, is preferably a change of at least 20% to 50%, more preferably by at least 50% to 75%, more preferably at least 75% to 100%, and more preferably 150% to 200%, and most preferably is a change of at least 2 to 10 fold compared to a control. Additionally, a change can be indicated by a change in binding specificity or substrate.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. For example, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260)2605-2608 (1985); Cassol et al. 1992; Rossolini et al. Mol. Cell Probes 8:91-98 (1994)). The term nucleic acid is used interchangeably with gene, cDNA, and mRNA encoded by a gene.

“Nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases. In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled with isotopes, chromophores, lumiphores, chromogens, or indirectly labeled such as with biotin to which a streptavidine complex may later bind. By assaying for the presence or absence of the probe, one can detect the presence or absence of the select sequence or subsequence.

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. Amino acids may be referred to herein by either their commonly known three letter symbols or by Nomenclature Commission. Nucleotides, likewise, may be referred to by their commonly accepted single-letter codes, i.e., the one-letter symbols recommended by the IUPAC-IUB.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA box element. A promoter also optionally includes distal enhancer or repressor elements which can be located as much as several thousand base pairs from the start site of transcription. A “constitutive” promoter is a promoter that is active under most environmental and developmental conditions. An “inducible” promoter is a promoter that is under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The Target Protein

The present invention provides for a nucleic acid encoding C. glabrata beta-tubulin or fragments thereof. Thus, in one aspect, the nucleic acids provided herein are defined by the proteins herein. The proteins provided herein comprise an amino acid sequence which has one or more of the following characteristics: greater than 70% sequence identity with SEQ ID NO:2, preferably greater than 80%, more preferably greater than 90%, more preferably greater than 95% or, in another embodiment, has 98 to 100% sequence identity with SEQ ID NO:2. As described above when describing the nucleotide in terms of SEQ ID NO: 1, the sequence identity may be slightly lower due to the degeneracy in the genetic code. Also included within the definition of the target proteins are amino acid sequence variants of wild-type target proteins.

Portions of the C. glabrata beta-tubulin nucleotide sequence may be used to identify polymorphic variants, orthologs, alleles, and homologues of the beta-tubulin. This identification can be made in vitro, e.g., under stringent hybridization conditions and sequencing, or by using the sequence information in a computer system for comparison with other nucleotide sequences. A preferred method used for identification is PCR (polymerase chain reaction). This amplification technique utilizes a thermostable polymerase to allow the dissociation of newly formed complimentary DNA and subsequent hybridization of primers to the target sequence with minimal loss of activity.

As will be appreciated by those in the art, the target proteins can be made in a variety of ways, including both synthesis de novo and by expressing a nucleic acid encoding the protein.

Target proteins of the present invention may also be modified in a way to form chimeric molecules comprising a fusion of a target protein with a tag polypeptide which provides an epitope to which an anti-tag antibody can selectively bind. The epitope tag is generally placed at the amino or carboxyl terminus of the target protein. Provision of the epitope tag enables the target protein to be readily detected, as well as readily purified by affinity purification. Various tag epitopes are well known in the art. Examples include polyhistidine (poly-his) or poly-histidine-glycine (poly-his-gly) tags; the flu HA tag polypeptide and its antibody 12CA5 (see, Field et al. (1988) Mol. Cell. Biol.:2159); the c-myc tag and the 8F9, 3C7, 6E10, G4, B7 and 9E10 antibodies thereto (see, Evans et al., (1985) Molecular and Cellular Biology, 5:3610); and the Herpes Simplex virus glycoprotein D (gD) tag and its antibody (see, Paborsky et al., (1990) Protein Engineering 3:547). Other tag polypeptides include the Flag-peptide (see, Hopp, et al. (1988) BioTechnology 6:1204); the Kt3 epitope peptide (see, Martine et al. (1992) Science, 255:192); tubulin epitope peptide (see, Skinner (1991) J. Biol. Chem. 266:15173); and the T7 gene 10 protein peptide tag (see, Lutz-Freyermuth et al. (1990) Proc. Natl. Acad. Sci. USA 87:6393).

The biological activity of any of the peptides provided herein can be routinely confirmed by assays such as those which assay microtubule binding activity. In one embodiment, polymorphic variants, alleles, and orthologs, homologues of the beta-tubulin protein are confirmed by using microtubule binding assays as known in the art.

The isolation of the biologically active C. glabrata beta-tubulin provides a means for assaying for modulators. Biologically active C. glabrata beta-tubulin is useful for identifying modulators of the beta-tubulin or fragments thereof, and can be used, for example, in binding assays including microtubule binding assays (Vale et al., Cell 42: 39-50 (1985)).

Isolation of the Gene Encoding Candida Glabrata Beta-Tubulin

General Recombinant DNA Methods

This invention relies on routine techniques in the field of recombinant genetics. Basic texts disclosing the general methods of use in this invention include Sambrook et al., Molecular Cloning, A Laboratory Manual (2^{nd} ed. 1989); Kriegler, Gene Transfer and Expression: A Laboratory Manual (1990); and Current Protocols in Molecular Biology (Ausubel et al., eds., 1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, mass spectrometry, sequenced proteins, derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1962 (1981), using an automated synthesizer, as described in Van Devanter et al., Nucleic Acids Res. 12:6159-6168 (1984). Purification of oligonucleotides is by either native acrylamide gel electrophoresis or by anion-exchange HPLC as described in Pearson & Reanier, J. Chrom. 225:137-149 (1983).

The sequence of the cloned genes and synthetic oligonucleotides can be verified after cloning using, e.g., the chain termination method for sequencing double-stranded templates of Wallace et al., Gene 16:21-26 (1981).

Cloning Methods for the Isolation of Candida Glabrata Beta-Tubulin

In general, the nucleic acid sequences encoding C. glabrata beta-tubulin and related nucleic acid sequence homologs are cloned from cDNA and genomic DNA libraries or isolated using amplification techniques with oligonucleotide primers. In a preferred instance, phosmid libraries are used. Alternatively, expression libraries can be used to clone the beta-tubulin and beta-tubulin homologues by detecting expressed homologues immunologically with antisera or purified antibodies made against the beta-tubulin that also recognize and selectively bind to the beta-tubulin homologue. Finally, amplification techniques using primers can be used to amplify and isolate the beta-tubulin from DNA or RNA. Amplification techniques using degenerate primers can also be used to amplify and isolate the beta-tubulin homologues. Amplification techniques using primers can also be used to isolate a nucleic acid encoding C. glabrata beta-tubulin. These primers can be used, e.g., to amplify a probe of several hundred nucleotides, which is then used to screen a library for full-length C. glabrata beta-tubulin.

Appropriate primers and probes for identifying the gene encoding homologues of C. glabrata beta-tubulin in other species are generated from comparisons of the sequences provided herein. As described above, antibodies can be used to identify the beta-tubulin homologues. For example, antibodies made to beta-tubulin or to a fragment thereof are useful for identifying C. glabrata beta-tubulin homologues.

Expression under appropriate (i.e., experimental growth) conditions may involve the generation of a genomic library, wherein the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is generally described in Grunstein et al., Proc. Natl. Acad. Sci. USA, 72:3961-3965 (1975).

An alternative method of isolating C. glabrata beta-tubulin nucleic acid and its homologues combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds. 1990)). Methods such as polymerase chain reaction and ligase chain reaction can be used to amplify nucleic acid sequences of the beta-tubulin directly from mRNA, from cDNA, from genomic libraries or cDNA libraries. Degenerate oligonucleotides can be designed to amplify the beta-tubulin homologues using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of the beta-tubulin encoding mRNA in physiological samples, for nucleic sequencing or for other purposes. Genes amplified by PCR can be purified from agarose gels and cloned into an appropriate vector.

Gene expression of C. glabrata beta-tubulin can also be analyzed by techniques known in the art, e.g., reverse transcription and amplification of mRNA, isolation of total RNA or poly A+RNA, northern blotting, dot blotting, in situ hybridization, RNase protection, quantitative PCR, and the like.

Synthetic oligonucleotides can be used to construct recombinant beta-tubulin genes for use as probes or for expression of protein. This method is performed using a series of overlapping oligonucleotides usually 40-120 bp in length, representing both the sense and nonsense strands of the gene. These DNA fragments are then annealed, ligated and cloned. Alternatively, amplification techniques can be used with precise primers to amplify a specific subsequence of the C. glabrata beta-tubulin gene. The specific subsequence is then ligated into an expression vector.

The gene for the modified beta-tubulin protein is typically cloned into intermediate vectors before transformation into prokaryotic or eukaryotic cells for replication and/or expression. The intermediate vectors are typically prokaryote vectors or shuttle vectors.

Expression Vector in Procaryotic Host Cell

To obtain high level expression of a cloned gene, such as those cDNAs encoding the C. glabrata beta-tubulin protein, it is important to construct an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al. Bacterial expression systems for expressing the beta-tubulin protein are available in, e.g., E. coli, Bacillus sp., and Salmonella (Palva et al., Gene 22:229-235 (1983); Mosbach et al., Nature 302:543-545 (1983)). Kits for such expression systems are commercially available. Eukaryotic expression systems for mammalian cells, yeast, and insect cells are well known in the art and are also commercially available. The pET expression system (Novagen) is a preferred prokaryotic expression system.

The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the C. glabrata beta-tubulin nucleic acid in host cells. A typical expression cassette thus contains a promoter operably linked to the nucleic acid sequence encoding the beta-tubulin and signals required for efficient polyadenylation of the transcript, ribosome binding sites, and translation termination. The nucleic acid sequence encoding the beta-tubulin may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23, and fusion expression systems such as GST and LacZ. Epitope tags can also be added to recombinant proteins to provide convenient methods of isolation, e.g., c-myc or histidine tags.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Transformation Methods

Standard transfection or transformation methods are used to produce bacterial, mammalian, yeast or insect cell lines that express large quantities of the C. glabrata beta-tubulin protein, which are then purified using standard techniques (see, e.g., Colley et al., J. Biol. Chem. 264:17619-17622 (1989); Guide to Protein Purification, in Methods in Enzymology, vol. 182 (Deutscher ed., 1990)).

Transformation of the prokaryotic host cell is performed according to standard techniques (see, e.g., Morrison, J. Bact., 132:349-351 (1977); Clark-Curtiss & Curtiss, Methods in Enzymology, 101:347-362 (Wu et al., eds, 1983). Any of the well known procedures for introducing foreign nucleotide sequences into host cells may be used. These include the use of calcium phosphate transfection, polybrene, protoplast fusion, electroporation, liposomes, microinjection, plasma vectors, viral Vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the C. glabrata beta-tubulin protein.

The media for culturing the transformants are well-known. For culturing E. coli, a nutrient medium such as LB medium or a minimal medium such as M9 medium is used with the addition of a carbon source, a nitrogen source, a vitamin source, etc. The transformant is cultured at approximately 16 to 42 degrees C. for 5-168 hours. The culture conditions may vary. E. coli cultures will be aerated on a shaker.

After the expression vector is introduced into the cells, the transfected cells are cultured under conditions favoring expression of the beta-tubulin protein, which is recovered from the culture using standard techniques.

Host Bacterial Strains

Numerous strains of E. coli exist which may serve as a host. Various E. coli strains are publicly available, such as E. coli K12 strain MM294 (ATCC 31, 446); E coli X1776 (ATCC 31,537); E. coli Strain W3110 (ATCC 27,325); and K5772 (ATCC 53,635). Preferred bacterial strains include E coli BL21 (DE3), BL21 (DE3), pLysS, BL21 (DE3) pLysEIV. Also used are derivatives of BL21 (DE3) codon plus (Stratagene), Rosetta (Novagen), and star strains (Stratagene). Codon plus and Rosetta express rare codons, resulting in better expression of human proteins in E. coli. The star strains have an RNAse gene deleted for higher mRNA stability and therefore, higher protein expression.

Purification of the Candida Glabrata Beta-Tubulin Protein

The C. glabrata beta-tubulin protein may be purified for use in functional assays. In a preferred embodiment, the protein is purified for use in assays to provide substantially pure samples. Alternatively, the protein need not be substantially pure as long as the sample comprising the target protein is substantially free of other components that can contribute to the production of ADP or phosphate.

The protein may be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography, chromatofocussing, selective precipitation with such substances as ammonium sulfate; and others (see, e.g., Scopes, Protein Purification: Principles and Practice (1982); U.S. Pat. No. 4,673,641; Ausubel et al. supra; and Sambrook et al., supra). For example, the target protein can be purified using a standard anti-target antibody column. Ultrafiltration and diafiltration techniques, in conjunction with protein concentration, are also useful. A preferred method of purification is use of Ni-NTA agarose (Qiagen).

The expressed protein can be purified by standard chromatographic procedures to yield a purified, biochemically active protein. The activity of any of the peptides provided herein can be routinely confirmed by the assays provided herein such as those which assay microtubule binding activity. Biologically active target protein is useful for identifying modulators of target protein or fragments (Kodama et al., J. Biochem. 99:1465-1472 (1986); Stewart et al., Proc. Nat'l Acad. Sci. USA 90:5209-5213 (1993)), and binding assays including microtubule binding assays (Vale et al., Cell 42:39-50 (1985)), as described in detail below.

Purification of the Candida Glabrata Beta-Tubulin Protein from Recombinant Bacteria

Recombinant proteins are expressed by transformed bacteria in large amounts, typically after promoter induction; but expression can be constitutive. Promoter induction with IPTG is a preferred method of expression. Bacteria are grown according to standard procedures in the art. Fresh or frozen bacteria cells are used for isolation of protein. Alternatively, it is possible to purify the beta-tubulin from bacteria periplasm. After the beta-tubulin is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO.sub.4 and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Standard Protein Separation Techniques for Purifying Candida Glabrata Beta-Tubulin Protein Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of the beta-tubulin protein can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

The Candida glabrata beta-tubulin protein can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Immunological Detection of the Candida Glabrata Beta-Tubulin Protein

In addition to the detection of the beta-tubulin genes and gene expression using nucleic acid hybridization technology, one can also use immunoassays to detect the C. glabrata beta-tubulin. Immunoassays can be used to qualitatively or quantitatively analyze the beta-tubulin. A general overview of the applicable technology can be found in Harlow & Lane, Antibodies: A Laboratory Manual (1988).

Antibodies to Candida Glabrata Beta-Tubulin

Methods of producing polyclonal and monoclonal antibodies that react specifically with the beta-tubulin are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546(1989)).

A number of C. glabrata beta-tubulin protein comprising immunogens may be used to produce antibodies specifically reactive with the beta-tubulin. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta-tubulin. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10.sup.4 or greater are selected and tested for their cross reactivity against non-beta-tubulin proteins or even other homologous proteins from other organisms (e.g., C. elegans unc-104 or human Kifl A), using a competitive binding immunoassay. Specific polyclonal antisera and monoclonal antibodies will usually bind with a K.sub.d of at least about 0.1 mM, more usually at least about 1 .mu.M, preferably at least about 0.1 .mu.M or better, and most preferably, 0.01 .mu.M or better.

Once C. glabrata beta-tubulin protein specific antibodies are available, the beta-tubulin can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio ed., 1980); and Harlow & Lane, supra.

Binding Assays

Antibodies can be used for treatment or to identify the presence of the C. glabrata beta-tubulin protein having the sequence identity characteristics as described herein. Additionally, antibodies can be used to identify modulators of the interaction between the antibody and the beta-tubulin as further described below. While the following discussion is directed toward the use of antibodies in the use of binding assays, it is understood that the same general assay formats such as those described for “non-competitive” or “competitive” assays can be used with any compound which binds to the beta-tubulin.

In a preferred embodiment, the C. glabrata beta-tubulin protein is detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology Volume 37: Antibodies in Cell Biology (Asai, ed. 1993); Basic and Clinical Immunology (Stites & Terr, eds., 7th ed. 1991): Immunological binding assays (or immunoassays) typically use an antibody that specifically binds to a protein or antigen of choice (in this case the beta-tubulin or antigenic subsequence thereof). The antibody may be produced by any of a number of means well known to those of skill in the art and as described above.

Other Assay Formats

Western blot (immunoblot) analysis is used to detect and quantify the presence of the beta-tubulin protein in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the beta-tubulin. The anti-beta-tubulin antibodies specifically bind to the beta-tubulin on the solid support. These antibodies may be directly labeled or alternatively may be subsequently detected using labeled antibodies (e.g., labeled sheep anti-mouse antibodies) that specifically bind to the anti-beta-tubulin antibodies. Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

Reduction of Non-specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Assays for Modulators of the Target Protein

Functional Assays

Assays that can be used to test for modulators of the target protein include a variety of in vitro or in vivo assays, e.g., microtubule depolymerization assays and cell based assays. (Kodama et al., J. Biochem. 99: 1465-1472 (1986); Stewart et al., Proc. Nat'l Acad. Sci. USA 90: 5209-5213 (1993); (Lombillo et al., J. Cell Biol. 128:107-115 (1995); (Vale et al., Cell 42:39-50 (1985)).

Modulation is tested by screening for candidate agents capable of modulating the activity of the target protein comprising the steps of combining a candidate agent with the target protein, as above, and determining an alteration in the biological activity of the target protein. Thus, in this embodiment, the candidate agent should both bind to the target protein (although this may not be necessary), and alter its biological or biochemical activity as defined herein. The methods include both in vitro screening methods and in vivo screening of cells for alterations in cell cycle distribution, cell viability, or for the presence, morphology, activity, distribution, or amount of mitotic spindles, as are generally outlined above.

In addition, target protein activity can be examined by determining modulation of target protein in vitro using cultured cells. The cells are treated with a candidate agent and the effect of such agent on the cells is then determined whether directly or by examining relevant surrogate markers. For example, characteristics such as mitotic spindle morphology and cell cycle distribution can be used to determine the effect.

Thus, in a preferred embodiment, the methods comprise combining a target protein and a candidate, and determining the effect of the candidate on the target protein. Generally, a plurality of assay mixtures are run in parallel with different agent concentrations to obtain a differential response to the various concentrations. Typically, one of these concentrations serves as a negative control, i.e., at zero concentration or below the level of detection.

As will be appreciated by those in the art, the components may be added in buffers and reagents to assay target protein activity and give optimal signals. Since the methods allow kinetic measurements, the incubation periods can be optimized to give adequate detection signals over the background.

Binding Assays

Competitive screening assays may be done by combining the target protein and a compound in a first sample in a first concentration. A level of activity is then determined for the protein. The protein is further contacted with the compound at a second concentration, and a level of activity of the protein is determined. A difference between the level of activity of the protein contacted with the first concentration of the compound and the level of activity of the protein contacted with the second concentration of the compound indicates that the compound is a modulator of protein activity.

Other Assay Components

The assays provided utilize target protein as defined herein. In one embodiment, portions of target protein are utilized; in a preferred embodiment, portions having target protein activity as described herein are used. In addition, the assays described herein may utilize either isolated target proteins or cells or animal models comprising the target proteins.

A variety of other reagents may be included in the screening assays. These include reagents like salts, neutral proteins, e.g. albumin, detergents, etc which may be used to facilitate optimal protein-protein binding and/or reduce non-specific or background interactions. Also, reagents that otherwise improve the efficiency of the assay, such as protease inhibitors, nuclease inhibitors, anti-microbial agents, etc.; may be used. The mixture of components may be added in any order that provides for the requisite binding.

Cell-Based Assays

A variety of cell-based assays may be used to determine activity, such as microtiter plate, disc plate diffusion, and inhibition of fungal hyphae length. ((R. N. Jones et al, Manual of Clinical Microbiology, 4^{th} ed., (1985); and M. A. Pfaller et al, Antimicrobial Agents and Chemotherapy, 34 (1990)). Assays that utilize techniques that are known in the art are the halo sensitivity/growth inhibition assay; and a subtractive screening assay, which may utilize a Saccharomyces cerevisiae strain and a C. glabrata strain modified to carry a mutant gene, as a basis of comparison.

### APPLICATIONS

The methods of the invention are used to identify compounds useful in the treatment of systemic fungal infections in mammals.

Candidate agents having the desired pharmacological activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-100 wt. %. The agents maybe administered alone or in combination with other treatments, i.e., radiation, or other chemotherapeutic agents.

In a preferred embodiment, the pharmaceutical compositions are in a water soluble form, such as pharmaceutically acceptable salts, which is meant to include both acid and base addition salts.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety.

1-12. (canceled)

13. An isolated protein, wherein (i) the protein can combine with alpha tubulin to form a heterodimer; and (ii) has a sequence that has greater than 90% amino acid sequence identity to SEQ ID NO:2 as measured using a sequence comparison algorithm.

14. The protein of claim 13 having a sequence which has greater than 95% sequence identity with SEQ ID NO:2.

15. The protein of claim 13 having a sequence which has greater than 98% sequence identity with SEQ ID NO:2.

16. An isolated protein of claim 13, wherein the protein specifically binds to polyclonal antibodies generated against a protein comprising SEQ ID NO: 2.

17. An isolated protein, wherein (i) the protein can combine with alpha tubulin to form a heterodimer; and (ii) has a sequence of SEQ ID NO:2.


SEQUENCE LISTING (3 sequences)

SEQ ID NO: 1 - DNA, 1758 bp, Candida glabrata
agaactgttttagttgttgaacttttttttgccattgaatagtctaacttaaccaaggagcaacatccaatgagagagattatccatatatctactggtcagtgtggtaaccagattggtgccgcattttgggaaactatctgtggtgaacatggcctggactataacggtaactaccatggaactgaggaaatccaaagatcgcgtctaggcgtttacttcaacgaggcctccagtggtaagtgggtcccaagatctatcaatgtcgatttggaaccaggtactatcgatgccgtgcgtacttctaacattggtaacttgtttagacctgacaactacatcttcggccaatcctctgctggtaacgtttgggctaaaggtcactacacagaaggtgccgaattggttgactccgtgatggacgtcattagacgtgaagccgaaggttgtgactctttgcaaggtttccagatcacacactccttgggtggtggtaccggttcaggtatgggtaccttactgatatctagaatcagagaagaatttcctgatagaatgatggcaacattctcagtcttgccatctccaaagacttctgacactgttgtcgaaccatataacgccaccctgtctgttcaccaattagtcgagtactcagatgaaactttctgtatcgataacgaggctctatacgaaatttgccaaacaactttgaagctgaatcaaccatcttacaatgacctaaataatcttgtgtcaagtgttatgtctggtgttaccacctctctacgttacccaggtcaattgaactccgatctgaggaaactggccgtcaacttggttcctttcccaagactgcatttcttcatggttggctatgcaccactaacagccattggctcccagtcctttagatcattgactgtgcctgagctaacccaacaaatgtttgactcaaagaatatgatggctgctgctgacccaagaaatggtagatatctaaccgtggcagcgttcttcagaggtaaagtttctgtcaaggaggtcgaagatgagatgcacaaggttcaaactaagaactcttcatattttgtcgagtggattccaaacaatgtacaaaccgctgtttgttctgttcctccaaaagatttggacatgtctgccacatttattggtaactctacctctatccaagaactatttaaaagagttggtaaccaattcaatgccatgtttaacagaaaggcgtttttacactggtatactagcgagggtatggatgaaatggaattcacagaggctcaagctaatatgaccgacttagtaaatgaatatcaacaataccaagaagctactgtggaggaagaaatagaggaaggtgctgagtacataacggaagaacagccaatgcctgaaacttttgattaattaaaaaaataattccaaaatatgacaactatttttcatttctaccacatctttttttatatctacagtcacagtgacctaaattcctttaacatcttttcaatttaatatcatgtttttctagctatatgatgcccaacctgtaagaaatgattccatagtgctaataatgttgaataggacattattataactaaatctatattatggaacaaatatttgtctgcctacaccaaatcgtgaattaaaatagaactatattaatagcgtcaagctcctgcttgattaagtgctttttgacatttgaattcctgaccctaatacctttct

SEQ ID NO: 2 - protein, 452 aa, Candida glabrata
MREIIHISTGQCGNQIGAAFWETICGEHGLDYNGNYHGTEEIQRSRLGVYFNEASSGKWVPRSINVDLEPGTIDAVRTSNIGNLFRPDNYIFGQSSAGNVWAKGHYTEGAELVDSVMDVIRREAEGCDSLQGFQITHSLGGGTGSGMGTLLISRIREEFPDRMMATFSVLPSPKTSDTVVEPYNATLSVHQLVEYSDETFCIDNEALYEICQTTLKLNQPSYNDLNNLVSSVMSGVTTSLRYPGQLNSDLRKLAVNLVPFPRLHFFMVGYAPLTAIGSQSFRSLTVPELTQQMFDSKNMMAAADPRNGRYLTVAAFFRGKVSVKEVEDEMHKVQTKNSSYFVEWIPNNVQTAVCSVPPKDLDMSATFIGNSTSIQELFKRVGNQFNAMFNRKAFLHWYTSEGMDEMEFTEAQANMTDLVNEYQQYQEATVEEEIEEGAEYITEEQPMPETFD

SEQ ID NO: 3 - DNA, 1356 bp, Candida glabrata
atgagagagattatccatatatctactggtcagtgtggtaaccagattggtgccgcattttgggaaactatctgtggtgaacatggcctggactataacggtaactaccatggaactgaggaaatccaaagatcgcgtctaggcgtttacttcaacgaggcctccagtggtaagtgggtcccaagatctatcaatgtcgatttggaaccaggtactatcgatgccgtgcgtacttctaacattggtaacttgtttagacctgacaactacatcttcggccaatcctctgctggtaacgtttgggctaaaggtcactacacagaaggtgccgaattggttgactccgtgatggacgtcattagacgtgaagccgaaggttgtgactctttgcaaggtttccagatcacacactccttgggtggtggtaccggttcaggtatgggtaccttactgatatctagaatcagagaagaatttcctgatagaatgatggcaacattctcagtcttgccatctccaaagacttctgacactgttgtcgaaccatataacgccaccctgtctgttcaccaattagtcgagtactcagatgaaactttctgtatcgataacgaggctctatacgaaatttgccaaacaactttgaagctgaatcaaccatcttacaatgacctaaataatcttgtgtcaagtgttatgtctggtgttaccacctctctacgttacccaggtcaattgaactccgatctgaggaaactggccgtcaacttggttcctttcccaagactgcatttcttcatggttggctatgcaccactaacagccattggctcccagtcctttagatcattgactgtgcctgagctaacccaacaaatgtttgactcaaagaatatgatggctgctgctgacccaagaaatggtagatatctaaccgtggcagcgttcttcagaggtaaagtttctgtcaaggaggtcgaagatgagatgcacaaggttcaaactaagaactcttcatattttgtcgagtggattccaaacaatgtacaaaccgctgtttgttctgttcctccaaaagatttggacatgtctgccacatttattggtaactctacctctatccaagaactatttaaaagagttggtaaccaattcaatgccatgtttaacagaaaggcgtttttacactggtatactagcgagggtatggatgaaatggaattcacagaggctcaagctaatatgaccgacttagtaaatgaatatcaacaataccaagaagctactgtggaggaagaaatagaggaaggtgctgagtacataacggaagaacagccaatgcctgaaacttttgat